(19) United States
(12) Patent Application Publication
(10) Pub. No.: US 2024/0093252 A1
(43) Pub. Date: Mar. 21, 2024

(54) ISOPROPYLMALATE SYNTHASE VARIANT AND A METHOD OF PRODUCING L-LEUCINE USING THE SAME

(71) Applicant: CJ CHEILJEDANG CORPORATION, Seoul (KR)

(72) Inventors: Hayun LEE, Seoul (KR); Ju Eun KIM, Seoul (KR); Ji Hye LEE, Seoul (KR)

(73) Assignee: CJ CHEILJEDANG CORPORATION, Seoul (KR)

(21) Appl. No.: 18/274,912

(22) PCT Filed: Jan. 28, 2022
(86) PCT No.: PCT/KR2022/001560

§ 371 Date: Jul. 28, 2023

(30) Foreign Application Priority Data
Mar. 5, 2021 (KR) 10-2021-0029469

(51) Int. Cl. C12P 13/06; C12N 9/10; C12N 15/77

(52) U.S. Cl. CPC C12P 13/06; C12N 9/1025; C12N 15/77; C12R 2001/15; C12Y 203/03013

(57) ABSTRACT

The present disclosure relates to an isopropylmalate synthase variant and a method of producing L-leucine using the same.

### TECHNICAL FIELD

The present disclosure relates to an isopropylmalate synthase variant, and a method of producing L-leucine using the same.

### BACKGROUND ART

L-Leucine is an essential amino acid, and is an expensive amino acid widely used in medicines, foods, feed additives, industrial chemicals. etc. It is mainly produced using microorganisms. Fermentation production of branched-chain amino acids, including L-leucine, is mainly carried out through microorganisms of the genus Escherichia or microorganisms of the genus Corynebacterium, and branched-chain amino acids are known to be biosynthesized from pyruvic acid via several steps using 2-ketoisocaproate as a precursor (Korean Patent No. 10-0220018, Korean Patent No. 10-0438146).

Isopropylmalate synthase, which is an enzyme involved in the biosynthesis of L-leucine, is an enzyme of the first step in the biosynthesis of leucine, which converts 2-ketoisovalerate, produced during the valine biosynthetic pathway, into isopropylmalate, which is needed in the biosynthesis of leucine instead of valine, and isopropylmalate synthase is an important enzyme in the process of leucine biosynthesis. However, isopropylmalate synthase is subjected to feedback inhibition by L-leucine, which is a final product, or derivatives thereof. Accordingly, although there is a variety of prior art related to isopropylmalate synthase variants which release feedback inhibition for the purpose of producing a high concentration of leucine (US Patent Publication No. 2015-0079641 and U.S. Pat. No. 6,403,342), research to discover better variants is still continuing.

### DISCLOSURE

### Technical Problem

The present inventors have endeavored to develop an isopropylmalate synthase variant which may be used for the production of L-leucine with a high concentration, and as a result, they developed a novel isopropylmalate synthase variant, and confirmed that L-leucine may be produced at a high yield from a microorganism including the same, thereby completing the present disclosure.

### Technical Solution

An object of the present disclosure is to provide a polypeptide variant having an isopropylmalate synthase activity.

Another object of the present disclosure is to provide a polynucleotide encoding the polypeptide variant of the present disclosure.

Still another object of the present disclosure is to provide a vector comprising the polynucleotide of the present disclosure.

Still another object of the present disclosure is to provide a microorganism of the genus Corynebacterium producing L-leucine, the microorganism comprising the polypeptide variant of the present disclosure; a polynucleotide encoding the same; or a vector including the same.

Still another object of the present disclosure is to provide a method of producing L-leucine, the method comprising a step of culturing, in a medium, a microorganism of the genus Corynebacterium producing L-leucine, the microorganism comprising the polypeptide variant of the present disclosure; a polynucleotide encoding the same; or a vector including the same.

Still another object of the present disclosure is to provide a composition for producing L-leucine, the composition comprising a Corynebacterium glutamicum strain including the polypeptide variant of the present disclosure or the polynucleotide of the present disclosure; or a medium in which the strain is cultured.

### Advantageous Effects

In the present disclosure, a polypeptide variant having an isopropylmalate synthase activity has an increased activity, as compared to a wild-type isopropylmalate synthase, and it may be applied to mass-production of L-leucine with high yield.

### DETAILED DESCRIPTION OF PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure.

Further, the scope of the present disclosure is not limited by the specific description described below. Further, those skilled in the art will recognize, or be able to ascertain using no more than routine experimentation, many equivalents to the specific embodiments of the disclosure described herein. Further, these equivalents should be interpreted to fall within the present disclosure.

To achieve the objects, one aspect of the present disclosure provides a polypeptide variant having an isopropylmalate synthase activity.

Specifically, the polypeptide variant may include one or more substitutions selected from the group consisting of i) a substitution of an amino acid residue corresponding to position 138 with another amino acid residue, ii) a substitution of an amino acid residue corresponding to position 162 with another amino acid residue. iii) a substitution of an amino acid residue corresponding to position 211 with another amino acid residue, iv) a substitution of an amino acid residue corresponding to position 245 with another amino acid residue, and v) a substitution of an amino acid residue corresponding to position 588 with another amino acid residue, in an amino acid sequence of SEQ ID NO: 1.

As used herein, the term “isopropylmalate synthase (IPMS)” refers to an enzyme that converts 2-ketoisovalerate into isopropylmalate, which is a precursor of L-leucine, by reacting with acetyl-CoA. In the present disclosure, the isopropylmalate synthase may be used interchangeably with isopropylmalate synthesizing enzyme, IPMS, LeuA protein, or LeuA.

In the present disclosure, a sequence of the LeuA may be obtained from GenBank of NCBI, which is a known database, and specifically, the LeuA may be a protein having an isopropylmalate synthase activity, which is encoded by leuA gene, but is not limited thereto.

The LeuA may be an enzyme derived from a microorganism of the genus Corynebacterium. Specifically, the LeuA may be an isopropylmalate synthase derived from Corynebacterium glutamicum.

The LeuA of the present disclosure may include the amino acid sequence of SEQ ID NO: 1, but is not limited thereto. Additionally, the LeuA may include a polypeptide having at least 80%, 90%, 95%, 96%, 97%, 98%, or 99% homology to the amino acid sequence of SEQ ID NO: 1. Further, it is obvious that an amino acid sequence having such homology or identity and exhibiting the activity corresponding to that of the isopropylmalate synthase may be included within the scope of the present disclosure even though it has an amino acid sequence in which some of the sequences are deleted, modified, substituted, or added.

For example, the LeuA may include those having addition or deletion of a sequence that do not alter the function of the protein of the present disclosure, at the N-terminus, C-terminus, and/or inside of the amino acid sequence, or a naturally occurring mutation, a silent mutation, or a conservative substitution.

The “conservative substitution” means substitution of one amino acid with another amino acid having similar structural and/or chemical properties. Such an amino acid substitution may generally occur based on similarity in the polarity, charge, solubility, hydrophobicity, hydrophilicity, and/or amphipathic nature of residues. Usually, conservative substitution may hardly affect or not affect activity of proteins or polypeptides.

The LeuA of the present disclosure may have the amino acid sequence of SEQ ID NO: 1 or an amino acid sequence having 90% or more identity thereto, or may consist of the amino acid sequence or may consist essentially of the amino acid sequence.

As used herein, the term “polypeptide variant” refers to a polypeptide which has an amino acid sequence different from that of the polypeptide variant before modification by conservative substitution and/or modification of one or more amino acids but maintains the functions or properties. Such a polypeptide variant may generally be identified by modifying one or more amino acids of the amino acid sequence of the polypeptide and evaluating the properties of the modified polypeptide. In other words, the ability of the polypeptide variant may be increased, unchanged, or decreased, as compared to that of the polypeptide before variation. Further, some polypeptide variants may include polypeptide variants in which one or more portions such as an N-terminal leader sequence or a transmembrane domain have been removed. Other polypeptide variants may include polypeptide variants in which a portion of the N- and/or C-terminus has been removed from the mature protein. The term “polypeptide variant” may be used interchangeably with terms such as modification, modified polypeptide, modified protein, mutant, mutein, and divergent, and is not limited thereto as long as it is a term used with the meaning of variation.

Further, the polypeptide variant may include deletions or additions of amino acids that have minimal effect on the properties and secondary structure of the polypeptide. For example, a signal (or leader) sequence that is co-translationally or post-translationally involved in the protein translocation may be conjugated to the N-terminus of the polypeptide variant. In addition, the polypeptide variant may be conjugated with other sequences or linkers so as to be identified, purified, or synthesized.

The polypeptide variant of the present disclosure may have an isopropylmalate synthase activity. Further, the polypeptide variant of the present disclosure may have the enhanced isopropylmalate synthase activity, as compared to the wild-type polypeptide having the isopropylmalate synthase activity.

The polypeptide variant of the present disclosure may include one or more substitutions selected from the group consisting of i) a substitution of an amino acid residue corresponding to position 138 with another amino acid residue, ii) a substitution of an amino acid residue corresponding to position 162 with another amino acid residue, iii) a substitution of an amino acid residue corresponding to position 211 with another amino acid residue, iv) a substitution of an amino acid residue corresponding to position 245 with another amino acid residue, and v) a substitution of an amino acid residue corresponding to position 588 with another amino acid residue in the amino acid sequence of SEQ ID NO: 1, specifically, one or more substitutions selected from the group consisting of i) a substitution of leucine, which is the amino acid residue corresponding to position 138, with another amino acid residue other than leucine, ii) a substitution of histidine, which is the amino acid residue corresponding to position 162, with another amino acid residue other than histidine, iii) a substitution of serine, which is the amino acid residue corresponding to position 211, with another amino acid residue other than serine, iv) a substitution of asparagine, which is the amino acid residue corresponding to position 245, with another amino acid residue other than asparagine, and v) a substitution of isoleucine, which is the amino acid residue corresponding to position 588, with another amino acid residue other than isoleucine in the amino acid sequence of SEQ ID NO: 1, and more specifically, one or more substitutions selected from the group consisting of i) a substitution of leucine, which is the amino acid residue corresponding to position 138, with glycine, ii) a substitution of histidine, which is the amino acid residue corresponding to position 162, with glutamate, iii) a substitution of serine, which is the amino acid residue corresponding to position 211, with leucine, iv) a substitution of asparagine, which is the amino acid residue corresponding to position 245, with serine, and v) a substitution of isoleucine, which is the amino acid residue corresponding to position 588, with proline in the amino acid sequence of SEQ ID NO: 1, and much more specifically, one or more, two or more, three or more, four or more, and five substitutions. The two or more substitutions may be a combination of i) and v); a combination of ii) and v): a combination of iii) and v): or a combination of iv) and v), but are not limited thereto. The four or more substitutions may be a combination of i), ii), iii), and iv), but are not limited thereto. The five or more substitutions may be a combination of i), ii), iii), iv) and v).

The polypeptide variant of the present disclosure may have/include an amino acid sequence of SEQ ID NO: 6 or SEQ ID NO: 8 or SEQ ID NO: 10 or SEQ ID NO: 12 or SEQ ID NO: 14. or may consist of/may consist essentially of the amino acid sequence of SEQ ID NO: 6 or SEQ ID NO: 8 or SEQ ID NO: 10 or SEQ ID NO: 12 or SEQ ID NO: 14. The polypeptide variant of the present disclosure may include a polypeptide having at least 80%, 90%, 95%, 96%, 97%, 98%, or 99% or more, and less than 100% identity or homology to the amino acid sequence of SEQ ID NO: 6 or SEQ ID NO: 8 or SEQ ID NO: 10 or SEQ ID NO: 12 or SEQ ID NO: 14, in which i) the amino acid residue corresponding to position 138 is glycine, ii) the amino acid residue corresponding to position 162 is glutamate, iii) the amino acid residue corresponding to position 211 is leucine, iv) the amino acid residue corresponding to position 245 is serine, or v) the amino acid residue corresponding to position 588 is proline in the amino acid sequence of SEQ ID NO: 6 or SEQ ID NO: 8 or SEQ ID NO: 10 or SEQ ID NO: 12 or SEQ ID NO: 14. Specifically, SEQ ID NO: 6 may be an amino acid sequence, in which leucine which is the amino acid residue corresponding to position 138 in the amino acid sequence of SEQ ID NO: 1 is substituted with glycine, SEQ ID NO: 8 may be an amino acid sequence, in which histidine which is the amino acid residue corresponding to position 162 is substituted with glutamate. SEQ ID NO: 10 may be an amino acid sequence, in which serine which is the amino acid residue corresponding to position 211 is substituted with leucine, SEQ ID NO: 12 may be an amino acid sequence, in which isoleucine which is the amino acid residue corresponding to position 588 is substituted with proline, and SEQ ID NO: 14 may be an amino acid sequence, in which asparagine which is the amino acid residue corresponding to position 245 is substituted with serine.

Further, it is obvious that a polypeptide variant having an amino acid sequence, in which some of the sequences are deleted, modified, substituted, conservatively substituted, or added, in addition to i) the position 138, ii) the position 162, iii) the position 211, iv) the position 245, or v) the position 588 in the amino acid sequence of SEQ ID NO: 6 or SEQ ID NO: 8 or SEQ ID NO: 10 or SEQ ID NO: 12 or SEQ ID NO: 14, is also included in the scope of the present disclosure, as long as the amino acid sequence has such identity or homology and exhibits the efficacy corresponding to that of the polypeptide variant of the present disclosure. Specifically, the substitution may include any one or more of (1) a variation (R558H) of substituting histidine for arginine which is the amino acid corresponding to position 558 of LeuA protein by substituting A for G which is a nucleotide at position 1673 of leuA gene encoding isopropylmalate synthase, (2) a variation (G561D) of substituting aspartic acid for glycine which is the amino acid corresponding to position 561 by substituting AT for GC which are nucleotides at positions 1682 and 1683 of leuA gene, or (3) a variation (P247C) of substituting cysteine for proline which is the amino acid at position 247 by substituting TG for CC which are nucleotides at positions 739 and 740 of leuA gene, and descriptions thereof are as described above.

More specifically, the polypeptide variant may include a polypeptide including variations (SEQ ID NO: 38) at positions 247, 558, and 561 in addition to i) the variation at position 138; or variations (SEQ ID NO: 40) at positions 247, 558, and 561 in addition to ii) the variation at position 162; or variations (SEQ ID NO: 42) at positions 247, 558, and 561 in addition to iii) the variation at position 211; variations (SEQ ID NO: 44) at positions 247, 558, and 561 in addition to iv) the variation at position 245; variations (SEQ ID NO: 46) at positions 247, 558, and 561 in addition to v) the variation at position 588; variations (SEQ ID NO: 48) at positions 247, 558, and 561 in addition to iii) the variation at position 211 and v) the variation at 588: variations (SEQ ID NO: 50) at positions 247, 558, and 561 in addition to i) the variation at position 138, ii) the variation at position 162, iii) the variation at position 211, and iv) the variation at position 245; or variations (SEQ ID NO: 52) at positions 247, 558, and 561 in addition to i) the variation at position 138, ii) the variation at position 162, iii) the variation at position 211, iv) the variation at position 245, and v) the variation at position 588, but is not limited thereto.

As used herein, the term “corresponding to” refers to amino acid residues at positions listed in the polypeptide, or amino acid residues that are similar, identical, or homologous to those listed in the polypeptide. Identifying the amino acid at the corresponding position may be determining a specific amino acid in a sequence that refers to a specific sequence. As used herein, “corresponding region” generally refers to a similar or corresponding position in a related protein or a reference protein.

For example, an arbitrary amino acid sequence is aligned with SEQ ID NO: 1. and based on this, each amino acid residue of the amino acid sequence may be numbered with reference to the amino acid residue corresponding to the amino acid residue of SEQ ID NO: 1. For example, a sequence alignment algorithm as described in the present disclosure may determine the position of an amino acid or the position at which modification such as substitution, insertion, or deletion occurs through comparison with that in a query sequence (also referred to as a “reference sequence”).

For such alignments, for example, the Needleman-Wunsch algorithm (Needleman and Wunsch, 1970, J. Mol. Biol. 48: 443-453). the Needleman program of EMBOSS package (EMBOSS: The European Molecular Biology Open Software Suite, Rice et al., 2000), Trends Genet. 16: 276-277) and the like may be used, but are not limited thereto, and a sequence alignment program, a pairwise sequence comparison algorithm, etc., known in the art, may be appropriately used.

As used herein, the term ‘homology’ or ‘identity’ means the degree of similarity between two given amino acid sequences or base sequences and may be expressed as a percentage. The terms ‘homology and identity’ may often be used interchangeably.

The sequence homology or identity of a conserved polynucleotide or polypeptide is determined by standard alignment algorithms, and the default gap penalty established by a program to be used may be used together. Substantially, homologous or identical sequences are generally capable of being hybridized with the entirety or a part of the sequence under moderately or highly stringent conditions. It is apparent that hybridization also includes hybridization of a polynucleotide with a polynucleotide including a general codon or a codon in consideration of codon degeneracy.

Whether any two polynucleotide or polypeptide sequences have homology, similarity, or identity may be determined using known computer algorithms such as the “FASTA” program, for example, using default parameters as in Pearson et al (1988) [Proc. Natl. Acad. Sci. USA 85]: 2444. Alternatively, the homology, similarity, or identity may be determined using Needleman-Wunsch algorithm (Needleman and Wunsch, 1970, J. Mol. Biol. 48: 443-453) as performed in the Needleman program of the EMBOSS package (EMBOSS: The European Molecular Biology Open Software Suite, Rice et al., 2000, Trends Genet. 16: 276-277) (version 5.0.0 or later) (including GCG program package (Devereux, J., et al, Nucleic Acids Research 12: 387 (1984)), BLASTP, BLASTN, FASTA (Atschul, [S,][F.,] [ET AL, J MOLEC BIOL 215]: 403 (1990); Guide to Huge Computers, Martin J. Bishop, [ED.,] Academic Press, San Diego, 1994, and [CARILLO ET AU.](1988) SIAM J Applied Math 48: 1073). For example, BLAST of the National Center for Biotechnology Information or ClustalW may be used to determine the homology, similarity, or identity.

The homology, similarity, or identity of polynucleotides or polypeptides may be determined by comparing sequence information using, for example, a GAP computer program such as Needleman et al. (1970), J Mol Biol. 48:443. as announced in, for example, Smith and Waterman, Adv. Appl. Math (1981) 2:482. In summary, the GAP program may be defined as the value acquired by dividing the number of similarly aligned symbols (namely, nucleotides or amino acids) by the total number of symbols in the shorter of two sequences. The default parameters for the GAP program may include (1) a binary comparison matrix (including values of 1 for identity and 0 for non-identity) and a weighted comparison matrix of Gribskov et al(1986) Nucl. Acids Res. 14: 6745 (or EDNAFULL (EMBOSS version of NCBI NUC4.4) substitution matrix) as disclosed in Schwartz and Dayhoff, eds., Atlas Of Protein Sequence And Structure, National Biomedical Research Foundation, pp. 353-358 (1979); (2) a penalty of 3.0 for each gap and an additional 0.10 penalty for each symbol in each gap (or gap opening penalty of 10, gap extension penalty of 0.5): and (3) no penalty for end gaps.

Another aspect of the present disclosure provides a polynucleotide encoding the polypeptide variant of the present disclosure.

As used herein, the term “polynucleotide” is a DNA or RNA strand having a certain length or more as a polymer of nucleotides in which nucleotide monomers are connected in a long chain by covalent bonds, and more specifically, it means a polynucleotide fragment encoding the protein variant.

The polynucleotide encoding the polypeptide variant of the present disclosure may include a nucleotide sequence encoding the amino acid sequence described by SEQ ID NO: 6 or SEQ ID NO: 8 or SEQ ID NO: 10 or SEQ ID NO: 12 or SEQ ID NO: 14 or SEQ ID NO: 38 or SEQ ID NO: 40 or SEQ ID NO: 42 or SEQ ID NO: 44 or SEQ ID NO: 46 or SEQ ID NO: 48 or SEQ ID NO: 50 or SEQ ID NO: 52, but is not limited thereto. Specifically, the polynucleotide of the present disclosure may have or include a nucleotide sequence of SEQ ID NO: 7 or SEQ ID NO: 9 or SEQ ID NO: 11 or SEQ ID NO: 13 or SEQ ID NO: 15 or SEQ ID NO: 39 or SEQ ID NO: 41 or SEQ ID NO: 43 or SEQ ID NO: 45 or SEQ ID NO: 47 or SEQ ID NO: 49 or SEQ ID NO: 51 or SEQ ID NO: 53.

In the polynucleotide, various modifications may be made in the coding region as long as the amino acid sequence of the polypeptide is not changed, in consideration of codon degeneracy or codons preferred in organisms that are intended to express the polypeptide. Specifically, the polynucleotide may consist of a nucleotide sequence having 80% or more, 90% or more, 95% or more, 96% or more, 97% or more, 98% or more, and less than 100% homology or identity to SEQ ID NO: 7 or SEQ ID NO: 9 or SEQ ID NO: 11 or SEQ ID NO: 13 or SEQ ID NO: 15 or SEQ ID NO: 39 or SEQ ID NO: 41 or SEQ ID NO: 43 or SEQ ID NO: 45 or SEQ ID NO: 47 or SEQ ID NO: 49 or SEQ ID NO: 51 or SEQ ID NO: 53, but is not limited thereto.

Further, the polynucleotide of the present disclosure may include a probe that may be prepared from a known gene sequence, for example, a sequence without limitation as long as it is a sequence that may hybridize with a complementary sequence to the entirety or a part of the polynucleotide sequence of the present disclosure under stringent conditions. The “stringent conditions” mean conditions that enable specific hybridization between polynucleotides.

These conditions are specifically described in documents (see J. Sambrook et al., Molecular Cloning, A Laboratory Manual, 2nd Edition, Cold Spring Harbor Laboratory press. Cold Spring Harbor, New York, 1989: F. M. Ausubel et al., Current Protocols in Molecular Biology, John Wiley & Sons, Inc., New York, 9.50-9.51, 11.7-11.8). Examples thereof include conditions in which polynucleotides having higher homology or identity, namely, polynucleotides having 70% or more, 75% or more, 80% or more, 85% or more, 90% or more, 95% or more, 96% or more, 97% or more, 98% or more, or 99% or more homology or identity are hybridized with each other while polynucleotides having lower homology or identity are not hybridized with each other, or washing conditions for common Southern hybridization, in which washing is performed once, specifically, two to three times at a salt concentration and temperature equivalent to 60° C. 1×SSC. 0.1% SDS, specifically 60° C., 0.1×SSC, 0.1% SDS, more specifically, 68° C., 0.1×SSC, 0.1% SDS.

Hybridization requires that two nucleic acids have complementary sequences, although mismatches between bases are allowed depending on the stringency of hybridization. The term “complementary” is used to describe the relation between nucleotide bases capable of being hybridized with each other. For example, with regard to DNA, adenine is complementary to thymine and cytosine is complementary to guanine. Therefore, the polynucleotide of the present disclosure may also include substantially similar nucleic acid sequences as well as isolated nucleic acid fragments that are complementary to the entire sequence.

Specifically, a polynucleotide having homology or identity to the polynucleotide of the present disclosure may be detected using hybridization conditions including a hybridization step at a Tm value of 55° C. and the above-described conditions. The Tm value may be 60° C., 63° C., or 65° C., but is not limited thereto, and may be appropriately adjusted by those skilled in the art according to the purpose.

The appropriate stringency to hybridize the polynucleotide depends on the length and degree of complementarity of the polynucleotide, and the variables are well known in the art (e.g., J. Sambrook et al., supra).

For example, the polynucleotide of the present disclosure may include any sequence without limitation as long as it encodes the amino acid sequence of SEQ ID NO: 6 or SEQ ID NO: 8 or SEQ ID NO: 10 or SEQ ID NO: 12 or SEQ ID NO: 14 or SEQ ID NO: 38 or SEQ ID NO: 40 or SEQ ID NO: 42 or SEQ ID NO: 44 or SEQ ID NO: 46 or SEQ ID NO: 48 or SEQ ID NO: 50 or SEQ ID NO: 52.

In the polynucleotide of the present disclosure, the polypeptide variant is as described in other aspects.

Still another aspect of the present disclosure provides a vector comprising the polynucleotide of the present disclosure.

The vector of the present disclosure refers to a DNA construct including a polynucleotide sequence encoding the polypeptide of interest operably linked to a suitable expression regulatory region (expression regulatory sequence) so that the polypeptide of interest may be expressed in a suitable host. The expression regulatory region may include a promoter capable of initiating transcription, any operator sequence for regulating the transcription. a sequence encoding a suitable mRNA ribosome binding site, and a sequence regulating termination of transcription and translation. The vector may be transformed into a suitable host cell and then replicated or function independently of the host genome, or may be integrated into the genome itself.

The vector used in the present disclosure is not particularly limited, but any vector known in the art may be used. Examples of commonly used vectors may include natural or recombinant plasmids, cosmids, viruses, and bacteriophages. For example, pWE15, M13, MBL3, MBL4, IXII, ASHII, APII, t10, t11, Charon4A, Charon21A, or the like may be used as a phage vector or a cosmid vector. pBR system, pUC system, pBluescript II system, pGEM system, pTZ system, pCL system, pET system, or the like may be used as a plasmid vector. Specifically. pDCM2(WO WO2021-187781 A1), pACYC177, pACYC184, pCL, pECCG117. pUC19, pBR322, pMW118, pCC1BAC vector or the like may be used.

For example, a polynucleotide encoding a polypeptide of interest may be inserted into a chromosome through a vector for intracellular chromosome insertion. Insertion of the polynucleotide into the chromosome may be performed by any method known in the art, for example, homologous recombination, but is not limited thereto. The vector may further include a selection marker for identifying the chromosome insertion. The selection marker is for selecting the cells transformed with vectors, i.e., for identifying the insertion of a nucleic acid molecule of interest, and markers that confer selectable phenotypes such as drug resistance, auxotrophy, resistance to cytotoxic agents, or expression of surface polypeptides may be used. In an environment treated with a selective agent, only cells expressing the selection marker survive or exhibit other phenotypic traits, and thus transformed cells may be selected.

As used herein, the term “transformation” means that a vector including a polynucleotide encoding a target protein is introduced into a host cell or a microorganism so that the protein encoded by the polynucleotide may be expressed in the host cell. The transformed polynucleotide may be located by being inserted into the chromosome of the host cell or located outside the chromosome as long as it may be expressed in the host cell. Further, the polynucleotide includes DNA and RNA encoding a protein of interest. The polynucleotide may be introduced in any form as long as it may be introduced into a host cell and then expressed. For example, the polynucleotide may be introduced into a host cell in the form of an expression cassette, which is a gene construct containing all elements required for self-expression. The expression cassette may usually include a promoter operably linked to the polynucleotide, a transcription termination signal, a ribosome binding site, and a translation termination signal. The expression cassette may be in the form of an expression vector capable of self-replicating. Further, the polynucleotide may be introduced into a host cell in its own form and operably linked to a sequence required for expression in the host cell, but is not limited thereto.

Further, as used herein, the term “operably linked” means that the polynucleotide sequence is functionally linked to a promoter sequence that initiates and mediates transcription of the polynucleotide encoding the protein variant of interest of the present disclosure.

In the vector of the present disclosure, the polynucleotide is as described in other aspects.

Still another aspect of the present disclosure provides a microorganism of the genus Corynebacterium producing L-leucine, the microorganism comprising the polypeptide variant of the present disclosure: the polynucleotide encoding the same; or the vector including the same.

As used herein, the term “microorganism” includes all of wild-type microorganisms or naturally or artificially genetically modified microorganisms, and it may be a microorganism in which a specific mechanism is weakened or strengthened due to insertion of a foreign gene or activity enhancement or inactivation of an endogenous gene, and may be a microorganism including a genetic modification for the production of the polypeptide, protein, or product of interest.

The microorganism of the present disclosure may be a microorganism including any one or more of the variant of the present disclosure, the polynucleotide of the present disclosure, and the vector including the polynucleotide of the present disclosure; a microorganism modified to express the variant of the present disclosure or the polynucleotide of the present disclosure; a microorganism (e.g., recombinant strain) expressing the variant of the present disclosure or the polynucleotide of the present disclosure: or a microorganism (e.g., recombinant strain) having the activity of the variant of the present disclosure, but is not limited thereto.

The microorganism of the present disclosure may be a microorganism naturally having the isopropylmalate synthase activity or the L-leucine-producing ability, or a microorganism prepared by expressing the polypeptide variant of the present disclosure in a parent strain having no isopropylmalate synthase activity or no L-leucine-producing ability, or by providing the L-leucine-producing ability for the parent strain, but is not limited thereto.

Specifically, the microorganism of the present disclosure may be a cell or microorganism expressing the polypeptide variant of the present disclosure by transforming with the polynucleotide of the present disclosure or the vector including the gene encoding the polypeptide variant of the present disclosure, and with respect to the objects of the present disclosure, the microorganism of the present disclosure may include all microorganisms capable of producing L-leucine by including the polypeptide variant of the present disclosure. For example, the microorganism of the present disclosure may be a recombinant microorganism having the enhanced L-leucine-producing ability, in which the polypeptide variant of the present disclosure is expressed by introducing the polynucleotide encoding the polypeptide variant of the present disclosure into a natural wild-type or L-leucine-producing microorganism. The recombinant microorganism having the enhanced L-leucine-producing ability may be a microorganism having the enhanced L-leucine-producing ability, as compared to the natural wild-type or unmodified microorganism, but is not limited thereto.

As used herein, the term “unmodified microorganism” does not exclude strains including mutations that may occur naturally in microorganisms, and may be a wild-type strain or a natural strain itself or may be a strain before the trait is changed by genetic variation due to natural or artificial factors. For example, the unmodified microorganism may be a strain into which the protein variant described in the present specification is not introduced or has not yet been introduced. The term “unmodified microorganism” may be used interchangeably with “strain before being modified”, “microorganism before being modified”, “unvaried strain”, “unmodified strain”, “unvaried microorganism”, or “reference microorganism”.

Specifically, the microorganism of the present disclosure may be Corynebacterium glutamicum, Corynebacterium crudilactis, Corynebacterium deserti. Corynebacterium efficiens, Corynebacterium callunae, Corynebacterium stationis, Corynebacterium singulare, Corynebacterium halotolerans, Corynebacterium stratum, Corynebacterium ammoniagenes, Corynebacterium pollutisoli, Corynebacterium imitans, Corynebacterium testudinoris, or Corynebacterium flavescens.

The microorganism of the present disclosure may be a microorganism including a nucleotide sequence encoding isopropylmalate synthase, in which one or more amino acid residues, excluding the amino acid residue i) at position 138, ii) at position 162, iii) at position 211, iv) at position 245, or v) at position 588 in the amino acid sequence of SEQ ID NO: 1 constituting the isopropylmalate synthase of the present disclosure, are substituted with another amino acid. Specifically, the substitution may include any one or more of (1) a variation (R558H) of substituting histidine for arginine which is the amino acid at position 558 of LeuA protein by substituting A for G which is a nucleotide at position 1673 of leuA gene encoding isopropylmalate synthase, (2) a variation (G561D) of substituting aspartic acid for glycine which is the amino acid at position 561 by substituting AT for GC which are nucleotides at positions 1682 and 1683 of leuA gene, or (3) a variation (P247C) of substituting cysteine for proline which is the amino acid at position 247 by substituting TG for CC which are nucleotides at positions 739 and 740 of leuA gene, and descriptions thereof are as described above.

Specifically, the microorganism producing L-leucine of the present disclosure may be a microorganism having the enhanced isopropylmalate synthase activity by expressing the isopropylmalate synthase including such a variation.

As used herein, the term “enhancement” of polypeptide activity means that the activity of a polypeptide is increased as compared to the intrinsic activity. The enhancement may be used interchangeably with terms such as up-regulation, overexpression, increase, etc. Here, the increase may include both exhibiting activity that was not originally possessed and exhibiting improved activity, as compared to the intrinsic activity or activity before modification. The “intrinsic activity” means activity of a specific polypeptide originally possessed by a parent strain before change of the trait or an unmodified microorganism when the trait is changed by genetic variation due to natural or artificial factors. This may be used interchangeably with “activity before modification”. The fact that the activity of a polypeptide is “enhanced” or “increased”, as compared to the intrinsic activity, means that the activity of a polypeptide is improved, as compared to the activity of a specific polypeptide originally possessed by a parent strain before change of the trait or an unmodified microorganism.

The enhancement may be achieved through the introduction of a foreign polypeptide or the enhancement of intrinsic activity of the polypeptide. The enhancement of activity of the polypeptide may be confirmed by an increase in the degree of activity and the expression level of the corresponding polypeptide or in the amount of a product produced from the corresponding polypeptide.

For the activity enhancement of the polypeptide, various methods well known in the art may be applied, and the method is not limited as long as the activity of the polypeptide of interest may be enhanced, as compared to that of the microorganism before being modified. Specifically, genetic engineering and/or protein engineering well known to those skilled in the art, which are routine methods of molecular biology, may be used, but the method is not limited thereto (e.g., Sitnicka et al. Functional Analysis of Genes. Advances in Cell Biology. 2010, Vol. 2. 1-16, Sambrook et al. Molecular Cloning 2012, etc.).

Specifically, the enhancement of the activity of the polypeptide of the present disclosure may be performed by:
1) increase in the intracellular copy number of the polynucleotide encoding the polypeptide;
2) replacement of a gene expression regulatory region on a chromosome encoding the polypeptide with a sequence exhibiting strong activity;
3) modification of a start codon of the gene encoding the polypeptide or a base sequence of a 5′-UTR region:
4) modification of the amino acid sequence of the polypeptide to enhance the activity of the polypeptide;
5) modification of the polynucleotide sequence encoding the polypeptide to enhance the activity of the polypeptide;
6) introduction of a foreign polynucleotide exhibiting the activity of the polypeptide;
7) codon optimization of the polynucleotide encoding the polypeptide;
8) analysis of the tertiary structure of the polypeptide to select the exposed site and to perform modification or chemical modification of the exposed site; or
9) a combination of 1) to 8), but is not particularly limited thereto.

More specifically, (1) the increase in the intracellular copy number of the polynucleotide encoding the polypeptide may be performed by introducing a vector, which replicates and functions irrespective of a host cell and is operably linked to the polynucleotide encoding the corresponding polypeptide, into a host cell. Alternatively, the increase may be achieved by the introduction of one copy or two or more copies of the polynucleotide encoding the corresponding polypeptide into a chromosome of a host cell. The introduction into the chromosome may be performed by introducing a vector capable of inserting the polynucleotide into a chromosome of a host cell into the host cell, but is not limited thereto. The vector is as described above.
2) The replacement of a gene expression control region (or expression control sequence) on a chromosome encoding a polypeptide with a sequence exhibiting strong activity may be, for example, occurrence of variation in a sequence due to deletion, insertion, non-conservative or conservative substitution, or a combination thereof, or replacement with a sequence exhibiting stronger activity so that the activity of the expression control region is further enhanced. The expression control region is not particularly limited thereto, but may include a promoter, an operator sequence, a sequence encoding a ribosome binding site, a sequence controlling the termination of transcription and translation, and the like. For example, the replacement may be to replace the original promoter with a strong promoter, but is not limited thereto.

Examples of known strong promoters include cj1 to cj7 promoters (U.S. Pat. No. 7,662,943 B2), lac promoter, trp promoter, trc promoter, tac promoter, lambda phage PR promoter, PL promoter, tet promoter, gapA promoter, SPL7 promoter, SPL13(sm3) promoter (U.S. patent Ser. No. 10/584,338 B2), 02 promoter (U.S. patent Ser. No. 10/273,491 B2), tkt promoter, yccA promoter, etc., but are not limited thereto.
3) The modification of a start codon of the gene encoding the polypeptide or a base sequence of a 5′-UTR region may be, for example, substitution with another start codon having a higher polypeptide expression rate, as compared to an endogenous start codon, but is not limited thereto.
4) and 5) The modification of the amino acid sequence or polynucleotide sequence may be occurrence of variation in the sequence due to deletion, insertion, nonconservative or conservative substitution of an amino acid sequence of the polypeptide or a polynucleotide sequence encoding the polypeptide or a combination thereof, or replacement with an amino acid sequence or polynucleotide sequence modified to have stronger activity or an amino acid sequence or polynucleotide sequence modified to be more active so that the activity of the polypeptide is enhanced, but is not limited thereto. The replacement may be specifically performed by inserting a polynucleotide into a chromosome by homologous recombination, but is not limited thereto. The vector used here may further include a selection marker for the confirmation of chromosome insertion. The selection marker is as described above.
6) The introduction of a foreign polynucleotide exhibiting the activity of the polypeptide may be the introduction of a foreign polynucleotide encoding a polypeptide exhibiting activity identical/similar to that of the polypeptide into a host cell. The foreign polynucleotide is not limited in its origin or sequence as long as it exhibits activity identical/similar to that of the polypeptide. The introduction may be performed by appropriately selecting a known transformation method by those skilled in the art. As the introduced polynucleotide is expressed in a host cell, a polypeptide may be produced, and the activity thereof may be increased.
7) The codon optimization of the polynucleotide encoding the polypeptide may be codon optimization of an endogenous polynucleotide so as to increase transcription or translation in a host cell or codon optimization of a foreign polynucleotide so as to perform optimized transcription and translation in a host cell.
8) The analysis of the tertiary structure of the polypeptide to select the exposed site and to perform modification or chemical modification of the exposed site may be, for example, to determine a template protein candidate according to the degree of similarity of the sequence by comparing the sequence information of a polypeptide to be analyzed with a database storing the sequence information of known proteins, to identify the structure based on this, and to select and to modify or chemically modify the exposed portion to be modified or chemically modified.

Such enhancement of the polypeptide activity may be an increase in the activity or concentration of the corresponding polypeptide, based on the activity or concentration of the polypeptide expressed in a wild-type or a microbial strain before being modified, or an increase in the amount of a product produced from the corresponding polypeptide, but is not limited thereto.

In the microorganism of the present disclosure, partial or entire modification of a polynucleotide (e.g., modification for coding the above-described protein variant) may be induced by (a) homologous recombination using a vector for chromosome insertion in the microorganism or genome editing using engineered nuclease (e.g., CRISPR-Cas9) and/or (b) treatment with light such as ultraviolet rays and radiation and/or chemicals, but is not limited thereto. A method of modifying a part or the entirety of the gene may include a method of using DNA recombination technology. For example, by introducing a nucleotide sequence or vector containing a nucleotide sequence homologous to the gene of interest into the microorganism to cause homologous recombination, a part or the entirety of the gene may be deleted. The introduced nucleotide sequence or vector may include a dominant selection marker, but is not limited thereto.

More specifically, the microorganism producing L-leucine of the present disclosure may be a microorganism further including a polypeptide including SEQ ID NO: 6 or SEQ ID NO: 8 or SEQ ID NO: 10 or SEQ ID NO: 12 or SEQ ID NO: 14 or SEQ ID NO: 38 or SEQ ID NO: 40 or SEQ ID NO: 42 or SEQ ID NO: 44 or SEQ ID NO: 46 or SEQ ID NO: 48 or SEQ ID NO: 50 or SEQ ID NO: 52. a polynucleotide encoding the polypeptide including SEQ ID NO: 6 or SEQ ID NO: 8 or SEQ ID NO: 10 or SEQ ID NO: 12 or SEQ ID NO: 14 or SEQ ID NO: 38 or SEQ ID NO: 40 or SEQ ID NO: 42 or SEQ ID NO: 44 or SEQ ID NO: 46 or SEQ ID NO: 48 or SEQ ID NO: 50 or SEQ ID NO: 52, or a polynucleotide including SEQ ID NO:7 or SEQ ID NO:9 or SEQ ID NO: 11 or SEQ ID NO: 13 or SEQ ID NO: 15 or SEQ ID NO: 39 or SEQ ID NO: 41 or SEQ ID NO: 43 or SEQ ID NO: 45 or SEQ ID NO: 47 or SEQ ID NO: 49 or SEQ ID NO: 51 or SEQ ID NO: 53.

In the microorganism of the present disclosure, the polypeptide variant, polynucleotide, vector, L-leucine, etc. are as described in other aspects.

Still another aspect of the present disclosure provides a method of producing L-leucine, the method comprising a step of culturing, in a medium, a microorganism of the genus Corynebacterium producing L-leucine, the microorganism comprising the polypeptide variant of the present disclosure; a polynucleotide encoding the same; or a vector including the same.

As used herein, the term “culture” means growing the microorganism of the genus Corynebacterium of the present disclosure under appropriately controlled environmental conditions. The culture process of the present disclosure may be performed according to suitable medium and culture conditions known in the art. Such a culture process may be easily adjusted and used by those skilled in the art according to the selected strain. Specifically, the culture may be a batch type, continuous type, and fed-batch type, but is not limited thereto.

As used herein, the term “medium” means a mixed substance containing nutrients required to culture the microorganism of the genus Corynebacterium of the present disclosure as a main component, and the medium supplies nutrients and growth factors, including water, which are indispensable for survival and development. Specifically, as the medium and other culture conditions used for culture of the microorganism of the genus Corynebacterium of the present disclosure, any one may be used without particular limitation as long as it is a medium used for common culture of microorganisms. The Corynebacterium glutamicum strain of the present disclosure may be cultured in a common medium containing proper carbon sources, nitrogen sources, phosphorus sources, inorganic compounds, amino acids and/or vitamins, etc., while controlling the temperature, pH, etc. under aerobic conditions. Specifically, the culture medium for the strain of the genus Corynebacterium may be found in the document [“Manual of Methods for General Bacteriology” by the American Society for Bacteriology (Washington D.C., USA, 1981)].

In the present disclosure, the carbon sources include carbohydrates such as glucose, saccharose, lactose, fructose, sucrose, maltose, etc.; sugar alcohols such as mannitol, sorbitol, etc., organic acids such as pyruvic acid, lactic acid, citric acid, etc.: amino acids such as glutamic acid, methionine, lysine, etc.; and the like. Natural organic nutrients such as starch hydrolysate, molasses, blackstrap molasses, rice bran, cassava, sugarcane residue, and corn steep liquor may be used. Specifically, carbohydrates such as glucose and sterilized pretreated molasses (i.e., molasses converted to reducing sugar) may be used, and appropriate amounts of other carbon sources may be used in various manners without limitation. These carbon sources may be used alone or in combination of two or more thereof, but are not limited thereto.

As the nitrogen sources, inorganic nitrogen sources such as ammonia, ammonium sulfate, ammonium chloride, ammonium acetate, ammonium phosphate, ammonium carbonate, ammonium nitrate, etc.; and organic nitrogen sources such as amino acids such as glutamic acid, methionine, glutamine, etc., peptone, NZ-amine, meat extract, yeast extract, malt extract, corn steep liquor, casein hydrolysate, fish or decomposition products thereof, and skim soybean cake or decomposition products thereof. etc. may be used. These nitrogen sources may be used alone or in combination of two or more thereof, but are not limited thereto.

The phosphorus sources may include monopotassium phosphate, dipotassium phosphate, or sodium-containing salts corresponding thereto. As the inorganic compounds, sodium chloride, calcium chloride, iron chloride, magnesium sulfate, iron sulfate, manganese sulfate, calcium carbonate, etc. may be used. In addition to these compounds, amino acids, vitamins and/or suitable precursors, etc. may be included. These components or precursors may be added to the medium batchwise or continuously, but is not limited thereto.

Further, during the culture of the Corynebacterium glutamicum strain of the present disclosure, pH of the medium may be adjusted by adding compounds such as ammonium hydroxide, potassium hydroxide, ammonia, phosphoric acid, or sulfuric acid to the medium in a proper manner. During the culture, foaming may be suppressed by using an antifoaming agent such as fatty acid polyglycol ester. Oxygen or oxygen-containing gas may be injected into the medium in order to maintain the aerobic state of the medium, or gas may not be injected or nitrogen. hydrogen, or carbon dioxide gas may be injected in order to maintain the anaerobic and microaerobic states, but is not limited thereto.

In the culture of the present disclosure, the culture temperature may be maintained at 20° C. to 45° C. specifically, at 25° C. to 40° C., and the strain may be cultured for about 10 hours to about 160 hours, but are not limited thereto.

L-leucine produced through the culture of the present disclosure may be secreted into the medium or may remain in the cells.

The method of producing L-leucine of the present disclosure may further include a step of preparing the Corynebacterium glutamicum strain of the present disclosure, or a step of preparing a medium for culture of the strain.

The method of producing L-leucine of the present disclosure may further include a step of recovering L-leucine from the medium according to the culture or from the Corynebacterium glutamicum strain of the present disclosure.

The recovery may be to collect L-leucine of interest by way of a suitable method known in the art according to the method of culturing the microorganism of the present disclosure, for example, a batch, continuous, or fed-batch culture method. For example, centrifugation, filtration, treatment with a crystallized protein precipitant (salting out), extraction, ultrasonic disintegration, ultrafiltration, dialysis, various forms of chromatography such as molecular sieve chromatography (gel filtration), adsorption chromatography, ion-exchange chromatography, and affinity chromatography, HPLC, or a combination thereof may be used. The L-leucine of interest may be recovered from the medium or microorganism by way of a suitable method known in the art.

Further, the method of producing L-leucine of the present disclosure may further include a purification step. The purification may be performed by way of a suitable method known in the art. For example, when the method of producing L-leucine of the present disclosure includes both the recovery step and the purification step, the recovery step and the purification step may be performed continuously or discontinuously regardless of the order, or may be performed simultaneously or by being combined into one step, but is not limited thereto.

In the method of the present disclosure, the polypeptide variant, polynucleotide. L-leucine, and the like are as described in other aspects.

Still another aspect of the present disclosure provides a composition for producing L-leucine, the composition comprising the Corynebacterium glutamicum strain including the polypeptide variant of the present disclosure or the polynucleotide of the present disclosure; or a medium in which the strain is cultured.

The composition of the present disclosure may further include arbitrary suitable excipients to be commonly used in compositions for producing amino acids. Such excipients may be, for example, a preservative, a wetting agent, a dispersing agent, a suspending agent, a buffering agent, a stabilizer, or an isotonic agent, but are not limited thereto.

In the composition of the present disclosure, the polypeptide variant, polynucleotide. L-leucine, and the like are as described in other aspects.

### MODE FOR CARRYING OUT THE INVENTION

Hereinafter, the present disclosure will be described in more detail with reference to exemplary embodiments. However, the following exemplary embodiments are only preferred embodiments for illustrating the present disclosure, and thus are not intended to limit the scope of the present disclosure thereto. Meanwhile, technical matters not described in the present specification can be sufficiently understood and easily implemented by those skilled in the technical field of the present disclosure or similar technical fields.

### Example 1. Construction of DNA Library Encoding Mutated Isopropylmalate Synthase

1-1. Construction of Vector Including leuA

To construct a leuA mutant library having an isopropylmalate synthase activity, a recombinant vector including leuA was first constructed. In order to amplify leuA gene (SEQ ID NO: 2) encoding LeuA protein (SEQ ID NO: 1, Uniprot accession code: P42455) derived from the wild-type Corynebacterium glutamicum, PCR was performed using the chromosome of the wild strain Corynebacterium glutamicum ATCC13032 as a template and primers of SEQ ID NOS: 3 and 4 by repeating 25 cycles consisting of denaturation at 94° C. for 1 minute, annealing at 58° C. for 30 seconds, and polymerization at 72° C. for 1 minute using Pfu DNA polymerase. Sequences of the used primers are as in Table 1 below.

TABLE 1

| SEQ ID NO. | Sequence name | Sequence (5′->3′) |
|---|---|---|
| SEQ ID NO: 3 | Primer 1 | TATGCTTCACCACATGACTTC |
| SEQ ID NO: 4 | Primer 2 | AAATCATTTGAGAAAACTCGAGG |

The PCR product was cloned into an E. coli vector pCR2.1 using a TOPO cloning kit (Invitrogen) to obtain ‘pCR-leuA’.

1-2. Construction of leuA Mutant Library

Based on the vector prepared in Example 1-1, a leuA mutant library was prepared using an error-prone PCR kit (clontech Diversify® PCR Random Mutagenesis Kit). A PCR reaction was performed using primers of SEQ ID NO: 3 and SEQ ID NO: 4 described in Table 1 under conditions where 0 to 3 mutations occur per 1000 bp.

In detail, PCR was performed by pre-heating at 94° C. for 30 seconds. followed by 25 cycles of denaturation at 94° C. for 30 seconds, and polymerization at 68° C. for 1 minute 30 seconds. The PCR product obtained at this time was subjected to 25 cycles of denaturation at 95° C. for 50 seconds, annealing at 60° C. for 50 seconds, and polymerization at 68° C. for 12 minutes using megaprimer (50 ng to 125 ng), followed by Dpnl treatment, and then transformed into E. col DH5a by a heat shock method, and plated on LB solid medium containing 25 mg/L of kanamycin. After selecting 20 types of transformed colonies. plasmids were obtained and sequenced. As a result, it was confirmed that mutations were introduced at different positions with a frequency of 2 mutations/kb. About 20,000 transformed E. coli colonies were taken and plasmids were extracted, which was named ‘pTOPO-leuA-library’.

### Example 2. Evaluation of Constructed Library and Selection of Mutants

2-1. Selection of Mutant Strains with Increased L-Leucine Production

The pTOPO-pheA-library prepared in Example 1-2 was transformed into the wild-type Corynebacterium glutamicum ATCC13032 by electroporation, and then spread on a nutrient medium (Table 2) containing 25 mg/L kanamycin to select 10,000 colonies of the strain into which mutant genes were inserted. Each selected colony was named ATCC13032/pTOPO_pheA(mt)1 to ATCC13032/pTOPO_pheA(mt) 10,000.

In order to identify colonies, in which production of L-leucine increased and production of L-phenylalanine among aromatic amino acids increased or decreased, among the obtained 10,000 colonies, fermentation titer was evaluated for each colony by the following method.

TABLE 2

| Type of medium | Ingredient |
|---|---|
| Production | 100 g of glucose, 40 g of (NH₄)₂SO₄, 2.5 g of soy |
| medium | protein, 5 g of corn steep solids, 3 g of urea, 1 g of |
| | KH₂PO₄, 0.5 g of MgSO₄•7H₂O, 100 μg of biotin, |
| | 1,000 μg of thiamine hydrochloride, 2000 μg of |
| | calcium-pantothenic acid, 3,000 μg of nicotinamide, |
| | 30 g of CaCO₃; (Based on 1 liter of distilled water), |
| | pH 7.0 |
| Nutrient | 10 g of glucose, 5 g of beef extract, 10 g of |
| medium | polypeptone, 2.5 g of sodium chloride, 5 g of yeast |
| | extract, 20 g of agar, 2 g of urea (based on 1 liter |
| | of distilled water) |

Each colony was inoculated using a platinum loop into a 250 ml corner-baffle flask containing 25 ug/ml of kanamycin in 25 ml of a production medium of Table 2, and then cultured at 30° C. for 60 hours under shaking at 200 rpm. After completion of the culture, L-leucine production was measured by a method of using high-performance liquid chromatography (HPLC, SHIMAZDU LC20A).

As a result, among 10.000 colonies, 5 kinds of strains (ATCC13032/pTOPO_leuA(mt)3847, ATCC13032/pTOPO_leuA(mt)4708, ATCC13032/pTOPO_leuA(mt)5109, ATCC13032/pTOPO_leuA(mt)7563, ATCC13032/pTOPO_leuA(mt)8459) showing the most improved L-leucine-producing ability, as compared to the wild-type Corynebacterium glutamicum ATCC13032, were selected. The concentrations of L-leucine produced in the selected strains are shown in Table 3 below.

TABLE 3

| Name of strain | L-leucine (g/L) |
|---|---|
| ATCC13032 | 0.87 |
| ATCC13032/pTOPO_leuA(mt)3847 | 1.23 |
| ATCC13032/pTOPO_leuA(mt)4708 | 1.27 |
| ATCC13032/pTOPO_leuA(mt)5109 | 1.39 |
| ATCC13032/pTOPO_leuA(mt)7563 | 1.19 |
| ATCC13032/pTOPO_leuA(mt)8459 | 1.25 |

As shown in Table 3, it was confirmed that Corynebacterium glutamicum ATCC13032/pTOPO_leuA(mt)3847 having a mutation in the leuA gene showed about 1.41-fold improvement in the L-leucine production, as compared to the parent strain, Corynebacterium glutamicum ATCC13032. It was also confirmed that ATCC13032/pTOPO_leuA(mt)4708, ATCC13032/pTOPO_leuA(mt)5109, ATCC13032/pTOPO_leuA(mt)7563, and ATCC13032/pTOPO_leuA(mt)8459 showed about 1.45-, 1.59-, 1.36-, and 1.38-fold improvement in the L-leucine production, as compared to the parent strain, respectively.

2-2. Identification of Mutations in Mutant Strains with Increased L-Leucine Production

In order to identify the leuA gene mutation of the selected 5 mutant strains, PCR was performed using DNA of each mutant strain as a template and primers of SEQ ID NO: 3 and SEQ ID NO: 4 described in Table 1 under conditions of denaturation at 94° C. for 5 minutes, followed by 30 cycles of denaturation at 94° C. for 30 seconds, annealing at 55° C. for 30 seconds, and polymerization at 72° C. for 1 minute and 30 seconds, and then polymerization at 72° C. for 5 minutes, and DNA sequencing was performed.

As a result of sequencing, in the ATCC13032/pTOPO_leuA(mt)3847 strain, both C and T at positions 412 and 413 of the leuA gene of SEQ ID NO: 2 were substituted with G, indicating that it encodes a variant (hereinafter, referred to as L138G) having a substitution of glycine for leucine, which is the amino acid at position 138 (at position 103 based on a literature, in which the translation start codon is read 35 backwards and the LeuA protein consists of 581 amino acids (SEQ ID NO: 5); hereinafter, referred to as only at position 138) of LeuA protein. The amino acid sequence of the LeuA variant (L138G) and the nucleotide sequence of the leuA variant encoding the same are as in SEQ ID NO: 6 and SEQ ID NO: 7.

It was confirmed that the ATCC13032/pTOPO_leuA(mt)4708 strain had a substitution of G for C, which are nucleotides at positions 484 and 486 of the leuA gene, indicating that it encodes a variant (hereinafter, referred to as H162E) having a substitution of glutamate for histidine, which is the amino acid at position 162 (at position 127 based on a literature, in which the translation start codon is read 35 backwards and the LeuA protein consists of 581 amino acids (SEQ ID NO: 5): hereinafter, referred to as only at position 162) of LeuA protein. The amino acid sequence of the LeuA variant (H162E) and the base sequence of the leuA variant encoding the same are as in SEQ ID NO: 8 and SEQ ID NO: 9.

It was confirmed that the ATCC13032/pTOPO_leuA(mt)5109 strain had a substitution of CTT for TCC, which are nucleotides at positions 631 to 633 of the leuA gene, indicating that it encodes a variant (hereinafter, referred to as S211L) having a substitution of leucine for serine, which is the amino acid at position 211 (at position 176 based on a literature, in which the translation start codon is read 35 backwards and the LeuA protein consists of 581 amino acids (SEQ ID NO: 5); hereinafter, referred to as only at position 211) of LeuA protein. The amino acid sequence of the LeuA variant (S211L) and the nucleotide sequence of the leuA variant encoding the same are as in SEQ ID NO: 10 and SEQ ID NO: 11.

It was confirmed that the ATCC13032/pTOPO_leuA(mt)7563 strain had a substitution of CC for AT, which are nucleotides at positions 1762 to 1763 of the leuA gene, indicating that it encodes a variant (hereinafter, referred to as 1588P) having a substitution of proline for isoleucine, which is the amino acid at position 588 (at position 553 based on a literature, in which the translation start codon is read 35 backwards and the LeuA protein consists of 581 amino acids (SEQ ID NO: 5); hereinafter, referred to as only at position 553) of LeuA protein. The amino acid sequence of the LeuA variant (1588P) and the nucleotide sequence of the leuA variant encoding the same are as in SEQ ID NO: 12 and SEQ ID NO: 13.

It was also confirmed that the ATCC13032/pTOPO_leuA(mt)8459 strain had a substitution of TC for AA, which are nucleotides at positions 733 to 734 of the leuA gene, indicating that it encodes a variant (hereinafter, referred to as N245S) having a substitution of serine for asparagine, which is the amino acid at position 245 (at position 210 based on a literature, in which the translation start codon is read 35 backwards and the LeuA protein consists of 581 amino acids (SEQ ID NO: 5): hereinafter, referred to as only at position 245) of LeuA protein. The amino acid sequence of the LeuA variant (N245S) and the nucleotide sequence of the leuA variant encoding the same are as in SEQ ID NO: 14 and SEQ ID NO: 15.

In the following Examples, it was examined whether the variations (L138G, H162E, S211L, N245S. 1588P) affect the L-leucine production of the microorganisms of the genus Corynebacterium.

### Example 3. Examination of L-Leucine-Producing Ability of Selected Mutant Strains

3-1. Construction of Insertion Vector Including leuA Variation

In order to introduce the variations selected in Example 2 into the strain, it was intended to construct an insertion vector. A site directed mutagenesis was used to construct a vector for introducing leuA (L138G, H162E, S211L, N245S, 1588P) variations. In detail, PCR was performed using the chromosome of the wild-type Corynebacterium glutamicum ATCC13032 strain as a template, and using a primer pair of SEQ ID NO: 16 and SEQ ID NO: 17, a primer pair of SEQ ID NO: 18 and SEQ ID NO: 19 for generating the L138G variation, and using a primer pair of SEQ ID NO: 16 and SEQ ID NO: 20, and a primer pair of SEQ ID NO: 19 and SEQ ID NO: 21 for generating the H162E variation. PCR was performed using a primer pair of SEQ ID NO: 16 and SEQ ID NO: 22, a primer pair of SEQ ID NO: 19 and SEQ ID NO: 23 for generating the S211L variation, and using a primer pair of SEQ ID NO: 16 and SEQ ID NO: 24, a primer pair of SEQ ID NO: 19 and SEQ ID NO: 25 for generating the N245S variation. PCR was performed using a primer pair of SEQ ID NO: 16 and SEQ ID NO: 26, a primer pair of SEQ ID NO: 19 and SEQ ID NO: 27 for generating the 1588P variation. In detail, PCR was performed by denaturation at 94° C. for 5 minutes, followed by 30 cycles consisting of denaturation at 94° C. for 30 seconds, annealing at 55° C. for 30 seconds, and polymerization at 72° C. for 1 minute and 30 seconds, and then polymerization at 72° C. for 5 minutes. Specific sequences of the used primers are shown in Table 4 below.

TABLE 4

| SEQ ID NO. | Sequence name | Sequence (5′->3′) |
|---|---|---|
| SEQ ID NO: 16 | Primer 3 | GGTCGACTCTAGAGGATCCCCTATGCTTCACCACATGACTTC |
| SEQ ID NO: 17 | Primer 4 | CAGGTGCTCACGAGCCTGAACCccAACCTGAATGGTGACATC |
| SEQ ID NO: 18 | Primer 5 | GACGATGTCACCATTCAGGTTggGGTTCAGGCTCGTGAG |
| SEQ ID NO: 19 | Primer 6 | GTGAATTCGAGCTCGGTACCCAAATCATTTGAGAAAACTCGAGGC |
| SEQ ID NO: 20 | Primer 7 | GATGGAGGTTGAGTTGTAGAAcTcCACGATAACGTTTTTTGCG |
| SEQ ID NO: 21 | Primer 8 | GGCGCAAAAAACGTTATCGTGgAgTTCTACAACTCAACCTCC |
| SEQ ID NO: 22 | Primer 9 | AGTGCCGGTGAAGGACTCAGGaagGTACTGCCAGCGCC |
| SEQ ID NO: 23 | Primer 10 | ACCAACTGGCGCTGGCAGTACcttCCTGAGTCCTTCACC |
| SEQ ID NO: 24 | Primer 11 | CATCTCAACGGTGGAACACAGGgaGATGATCATTGGGTTCTC |
| SEQ ID NO: 25 | Primer 12 | CCTGAGAACCCAATGATCATCtcCCTGTGTTCCACCG |
| SEQ ID NO: 26 | Primer 13 | TGCCTTCAGCGAAGCGTAGGTGggGGAGCCAGCGATGC |
| SEQ ID NO: 27 | Primer 14 | GGCGTCGGCATCGCTGGCTCCccCACCTACGCTTCGCTG |

Cloning was performed by fusing the PCR product with a linear pDCM2 vector digested with Smal restriction enzyme, using In-Fusion enzyme through the homologous sequence of the terminal 15 bases between the DNA fragments, thereby constructing ‘pDCM2-leuA(L138G)’, ‘pDCM2-leuA(H162E)’, ‘pDCM2-leuA(S211L)’, ‘pDCM2-leuA(N245S)’, and ‘pDCM2-leuA(1588P)’ which are vectors for substituting the amino acids of LeuA. Further, ‘pDCM2-leuA(S211L, 1588P), pDCM2-leuA(L138G, H162E, S211L, N245S)’, ‘pDCM2-leuA(L138G, H162E, S211L, N245S, 1588P)’, which are vectors for substituting the amino acids of LeuA, were constructed according to combination of the variants.

3-2. Introduction of Variant into Corynebacterium glutamicum ATCC13032 Strain and Evaluation

pDCM2-leuA(L138G), pDCM2-leuA(H162E), pDCM2-leuA(S211L). pDCM2-leuA(N245S), pDCM2-leuA(1588P), pDCM2-leuA(S211L, 1588P), pDCM2-leuA(L138G, H162E, S211L, N245S), pDCM2-leuA(L138G, H162E, S211L, N245S, 1588P) vectors prepared in Example 3-1 were transformed into Corynebacterium glutamicum ATCC13032 strain by electroporation, respectively and the strains, in which each vector was inserted on the chromosome by recombination of the homologous sequence, were selected in a medium containing 25 mg/L of kanamycin. The selected primary strains were again subjected to secondary crossover, and strains into which the target gene variation was introduced were selected. Finally, whether or not the leuA gene variation was introduced into the transformed strain was confirmed by performing PCR using primers of SEQ ID NO: 3 and SEQ ID NO: 4, and then analyzing the nucleotide sequence, thereby identifying that the variation was introduced into the strain. A total of 8 strains were prepared, and named ‘ATCC13032_leuA_L138G’, ‘ATCC13032_leuA_H162E’. ‘ATCC13032_leuA_S211L’, ‘ATCC13032_leuA_N245S’, ‘ATCC13032_leuA_1588P’, ‘ATCC13032_leuA_(S211L, 1588P)’‘ATCC13032_leuA_(L138G, H162E, S211 L, N245S)’, ‘ATCC13032_leuA_(L138G, H162E, S211L, N245S, 1588P)’, respectively.

In order to evaluate L-leucine-producing ability of a total of 8 strains thus prepared, a flask fermentation titer was evaluated. Each one platinum loop of the parent strain Corynebacterium glutamicum ATCC13032 and the prepared ATCC13032_leuA_L138G, ATCC13032_leuA_H162E, ATCC13032_leuA_S211 L, ATCC13032_leuA_N245S, ATCC13032_leuA_1588P, ATCC13032_leuA (S211L, 1588P), ATCC13032_leuA_(L138G, H162E, S211L, N245S), ATCC13032_leuA_(L138G, H162E, S211L, N245S, 1588P) was inoculated into a 250 ml corner-baffle flask containing 25 ml of a production medium, and then cultured at 30° C. for 60 hours under shaking at 200 rpm to produce L-leucine. After completion of the culture, L-leucine production was measured by HPLC. The concentration of leucine in the culture medium of each tested strain is shown in Table 5 below.

TABLE 5

| Name of strain | Leucine (g/L) |
|---|---|
| ATCC13032 | 0.87 |
| ATCC13032_leuA_L138G | 1.27 |
| ATCC13032_leuA_H162E | 1.30 |
| ATCC13032_leuA_S211L | 1.38 |
| ATCC13032_leuA_N245S | 1.22 |
| ATCC13032_leuA_I588P | 1.20 |
| ATCC13032_leuA_(S211L, I588P) | 1.32 |
| ATCC13032_leuA_(L138G, H162E, | 1.36 |
| S211L, N245S) | |
| ATCC13032_leuA_(L138G, H162E, | 1.35 |
| S211L, N245S, I588P) | |

As shown in Table 5, ATCC13032_leuA_L138G with L138G variation in the leuA gene showed about 1.45-fold improvement in the L-leucine yield, as compared to the parent strain Corynebacterium glutamicum ATCC13032, ATCC13032_leuA_H162E with H162E variation showed about 1.49-fold improvement in the L-leucine yield, as compared to the parent strain Corynebacterium glutamicum ATCC13032, ATCC13032_leuA_S211L with S211L variation showed about 1.58-fold improvement in the L-leucine yield, as compared to the parent strain Corynebacterium glutamicum ATCC13032, ATCC13032_leuA_N245S with N245S variation showed about 1.40-fold improvement in the L-leucine yield, as compared to the parent strain Corynebacterium glutamicum ATCC13032, ATCC13032_leuA_1588P with 1588P variation showed about 1.37-fold improvement in the L-leucine yield, as compared to the parent strain Corynebacterium glutamicum ATCC13032, and ATCC13032_leuA_(S211L, 1588P) showed about 1.51-fold improvement in the L-leucine yield, as compared to the parent strain. ATCC13032_leuA_(L138G, H162E, S211L, N245S) and ATCC13032_leuA_(L138G, H162E, S211L, N245S, 1588P) showed about 1.56-fold improvement in the L-leucine yield, as compared to the parent strain Corynebacterium glutamicum.

### Example 4. Examination of Leucine-Producing Ability of Selected leuA Variations in Leucine-Producing Strains

The wild-type strain of the genus Corynebacterium produces only trace amounts of leucine even though it produces leucine. Accordingly, a leucine-producing strain derived from the wild-type Corynebacterium glutamicum ATCC13032 was prepared, and the selected variations were introduced to perform an experiment for examining the leucine-producing ability. The detailed experimental method and results are as follows.

4-1. Preparation of L-Leucine-Producing CJL-8109 Strain

As strains for producing high concentrations of L-leucine, the wild-type Corynebacterium glutamicum ATCC13032-derived strains were prepared, each including (1) a variation (R558H), in which histidine was substituted for arginine which is an amino acid at position 558 of LeuA protein by substituting A for G which is a nucleotide at position 1673 of leuA gene, (2) a variation (G561D), in which aspartic acid was substituted for glycine which is an amino acid at position 561 of LeuA protein by substituting AT for GC which are nucleotides at positions 1682 and 1683 of leuA gene, or (3) variation (P247C), in which cysteine was substituted for proline which is an amino acid at position 247 of LeuA protein by substituting TG for CC which are nucleotides at positions 739 and 740 of leuA gene.

In detail, pDCM2-leuA(R558H, G561D) vector (US Patent Publication NO. 2021-0254111) including the leuA gene variations (R558H. G561D) was transformed into Corynebacterium glutamicum ATCC13032 by electroporation. and strains in which the vector was inserted on the chromosome by recombination of homologous sequence were selected in a medium containing 25 mg/L kanamycin. The selected primary strains were again subjected to secondary crossover, and strains into which the leuA gene variation was introduced were selected. Finally, whether or not the variation was introduced into the transformed strain was confirmed by performing PCR (94° C. for 5 minutes, followed by 30 cycles of 94° C. for 30 seconds/55° C. for 30 seconds/72° C. for 90 seconds, and 72° C. for 5 minutes) using primers of SEQ ID NOS. 28 and 55, and then analyzing the nucleotide sequence, thereby identifying introduction of R558H, G561D variations. Specific sequences of the used primers are shown in Table 6 below. ATCC13032_leuA_(R558H, G561D) strain transformed with the pDCM2-leuA(R558H, G561D) vector was named ‘CJL-8100’.

TABLE 6

| SEQ ID NO. | Sequence name | Sequence (5′->3′) |
|---|---|---|
| SEQ ID NO: 28 | Primer 15 | AACACGACCGGCATCCCGTCGC |
| SEQ ID NO: 29 | Primer 16 | AAATCATTTGAGAAAACTCGAGG |
| SEQ ID NO: 19 | Primer 6 | GTGAATTCGAGCTCGGTACCCAAATCATTTGAGAAAACTCGAGGC |
| SEQ ID NO: 54 | Primer 27 | GGTGATCATCTCAACGGTGGAACACAGGTTGATGATCATTGGGTT |
| SEQ ID NO: 55 | Primer 28 | AACCCAATGATCATCAACCTGTGTTCCACCGTTGAGATGATCACC |

To introduce the variation (P247C) into the L-leucine-producing strain, CJL-8100, an insertion vector was constructed.

In detail, PCR was performed using the chromosome of CJL-8100 strain as a template and primer pairs of SEQ ID NOS: 28 and 29 and SEQ ID NOS: 54 and 55. PCR was performed as follows: denaturation at 94° C. for 5 minutes, 30 cycles of at 94° C. for 30 seconds, at 55° C. for 30 seconds, and at 72° C. for 1 minute 30 seconds, followed by polymerization at 72° C. for 5 minutes. The resulting PCR product was cloned into a linear pDCM2 vector digested with Smal restriction enzyme using In-Fusion enzyme through fusion of the homologous sequence of the terminal 15 bases between DNA fragments, thereby constructing a pDCM2-leuA(P247C, R558H, G561D) vector including the leuA variation encoding the LeuA variant, in which histidine was substituted for arginine which is an amino acid at position 558 of the LeuA amino acid sequence of the wild-type strain, and aspartic acid was substituted for glycine which is an amino acid at position 561 thereof, and cysteine (Cys) was substituted for proline(Pro) which is an amino acid at position 247 of LeuA.

The pDCM2-leuA(P247C, R558H, G561D) vector was transformed into the wild-type Corynebacterium glutamicum ATCC13032 by electroporation, and strains in which the vector was inserted on the chromosome by recombination of homologous sequence were selected in a medium containing 25 mg/L kanamycin. The selected primary strains were again subjected to secondary crossover, and strains into which the leuA gene variations were introduced were selected. Finally, whether or not the variations were introduced into the transformed strain was confirmed by performing PCR (94° C. for 5 minutes, 30 cycles of denaturation at 94° C. for 30 seconds, annealing at 55° C. for 30 seconds, and polymerization at 72° C. 90 seconds, followed by polymerization at 72° C. for 5 minutes) using primers of SEQ ID NOS: 3 and 4, and then analyzing the nucleotide sequence, thereby identifying introduction of P247C, R558H, and G561D variations. The ATCC13032_leuA_(P247C, R558H, G561D) strain transformed with the pDCM2-leuA(P247C, R558H, G561D) vector was named ‘CA13-8105’.

CA13-8105 was deposited at the Korean Culture Center of Microorganisms, an international depository authority under the Budapest Treaty, on Apr. 29, 2020, and assigned Accession No. KCCM12709P.

To increase the L-leucine productivity in the prepared CA13-8105 strain, a strain into which ilvE variant(V156A) encoding branched-chain amino acid aminotransferase was introduced was prepared (WO WO2021-112469 A1). In detail, the pDCM2-ilvE(V156A) vector including the ilvE gene variation was transformed into Corynebacterium glutamicum CJL-8100 by electroporation, and strains in which the vector was inserted on the chromosome by recombination of homologous sequence were selected in a medium containing 25 mg/L kanamycin. The selected primary strains were again subjected to secondary crossover, and strains into which the ilvE gene variation was introduced were selected. Finally, whether or not the variation was introduced into the transformed strain was confirmed by performing PCR (94° C. for 5 minutes, 30 cycles of 94° C. 30 seconds/55° C. 30 seconds/72° C. 90 seconds, followed by 72° C. for 5 minutes) using primers of SEQ ID NOS: 30 and 31 of Table 7 below, and then analyzing the nucleotide sequence, thereby identifying introduction of V156A variation. The strain transformed with the pDCM2-ilvE(V156A) vector was named ‘CJL-8108’.

TABLE 7

| SEQ ID NO. | Sequence name | Sequence (5′->3′) |
|---|---|---|
| SEQ ID NO: 30 | Primer 23 | GTCACCCGATCGTCTGAAG |
| SEQ ID NO: 31 | Primer 24 | GTCTTAAAACCGGTTGAT |

To increase the L-leucine productivity in the prepared CJL-8108 strain, a strain into which gltA variant(M3121) with weakened citrate synthase activity was introduced was prepared.

In detail, site directed mutagenesis was used in the construction of a vector for introducing the gltA(M3121) variation. PCR was performed using the chromosome of the wild-type Corynebacterium glutamicum ATCC13032 as a template and primers of Table 8 below. PCR was performed under conditions of denaturation at 94° C. for 5 minutes, 30 cycles of at 94° C. for 30 seconds, at 55° C. for 30 seconds, and at 72° C. for 1 minute and 30 seconds, followed by polymerization at 72° C. for 5 minutes. The resulting gene fragment was cloned into a linear pDCM2 vector digested with Smal restriction enzyme using In-Fusion enzyme through fusion of the homologous sequence of the terminal 15 bases between DNA fragments, thereby constructing a pDCM2-gltA(M3121) vector for substituting methionine at position 312 with isoleucine.

TABLE 8

| SEQ ID NO. | Sequence name | Sequence (5′->3′) |
|---|---|---|
| 32 | gltA M312I Up F | GTGAATTCGAGCTCGGTACCCGCGGGAATCCTGCGTTA |
| | | CCGC |
| 33 | gltA M312I Up R | TGTAAACGCGGTGTCCGAAGCCGATGAGGCGGACGCC |
| | | GTCTT |
| 34 | gltA M312I Down F | AAGACGGCGTCCGCCTCATCGGCTTCGGACACCGCGT |
| | | TTACA |
| 35 | gltA M312I Down R | GGTCGACTCTAGAGGATCCCCTTAGCGCTCCTCGCGAG |
| | | GAAC |

The pDCM2-gltA(M3121) vector including the gltA gene variation was transformed into Corynebacterium glutamicum CJL-8108 by electroporation, and strains in which the vector was inserted on the chromosome by recombination of homologous sequence were selected in a medium containing 25 mg/L kanamycin. The selected primary strains were again subjected to secondary crossover, and strains into which the gltA gene variation was introduced were selected. Finally, whether or not the variation was introduced into the transformed strain was confirmed by performing PCR (94° C. for 5 minutes, 30 cycles of 94° C. 30 seconds/55° C. 30 seconds/72° C. 90 seconds, followed by 72° C. for 5 minutes) using primers of SEQ ID NOS: 36 and 37 of Table 9 below, and then analyzing the nucleotide sequence, thereby identifying introduction of M3121 variation. The strain transformed with the pDCM2-gltA(M3121) vector was named ‘CJL-8109’.

TABLE 9

| SEQ ID NO. | Sequence name | Sequence (5′->3′) |
|---|---|---|
| SEQ ID NO: 36 | Primer 25 | CAATGCTGGCTGCGTACGC |
| SEQ ID NO: 37 | Primer 26 | CTCCTCGCGAGGAACCAACT |

4-2. Construction of Insertion Vector Including leuA Variation

In order to introduce the variations (L138G. H162E, S211 L, N245S, 1588P) selected in Example 2 into the L-leucine producing strain CJL-8109 prepared in Example 4-1, it was intended to construct an insertion vector.

PCR was performed using the chromosome of the CJL-8109 strain as a template, and a primer pair of Table 4. PCR was performed under conditions of denaturation at 94° C. for 5 minutes, 30 cycles of denaturation at 94° C. for 30 seconds, annealing at 55° C. for 30 seconds, and polymerization at 72° C. for 1 minute and 30 seconds, followed by polymerization at 72° C. for 5 minutes. The resulting PCR product was cloned into a linear pDCM2 vector digested with Smal restriction enzyme using In-Fusion enzyme through fusion of the homologous sequence of the terminal 15 bases between DNA fragments, thereby constructing a total of 8 vectors, ‘pDCM2-leuA(L138G, P247C, R558H, G561D)’, ‘pDCM2-leuA(H162E, P247C, R558H, G561D)’, ‘pDCM2-leuA(S211L, P247C, R558H, G561D)’, ‘pDCM2-leuA(N245S, P247C, R558H, G561D)’, ‘pDCM2-leuA(P247C, R558H, G561D, 1588P)’, ‘pDCM2-leuA(S211L. P247C, 1588P)’, ‘pDCM2-leuA(L138G, H162E, S211L, N245S, P247C, R558H, G561D)’, and ‘pDCM2-leuA(L138G, H162E, S211L, N245S, P247C, R558H, G561D, 1588P)’.

4-3. Introduction of leuA Variant into CJL-8109 Strain and Evaluation

The L-leucine producing strain CJL-8109 was transformed with each of the vectors prepared in Example 4-2, and the strains, in which each vector was inserted on the chromosome by recombination of the homologous sequence, were selected in a medium containing 25 mg/L of kanamycin. The selected primary strains were again subjected to secondary crossover, and strains into which the target gene variation was introduced were selected. Finally, whether or not the leuA gene variation was introduced into the transformed strain was confirmed by performing PCR using primers of SEQ ID NO: 3 and SEQ ID NO: 4, and then analyzing the nucleotide sequence, thereby identifying that the leuA variation was introduced into the strain. A total of 8 strains thus prepared were named as in Table 11 below, and the amino acid sequence of the variant including the variation and the nucleotide sequence of the leuA variant encoding the same are shown in Table 10 below.

TABLE 10

| | No. of | |
|---|---|---|
| Name of strain | strain | SEQ ID NO. |
| CJL-8109_leuA_L138G, P247C, R558H, | CJL-8117 | SEQ ID NO: |
| G561D | | 38, 39 |
| CJL-8109_leuA_H162E, P247C, R558H, | CJL-8118 | SEQ ID NO: |
| G561D | | 40, 41 |
| CJL-8109_leuA_S211L, P247C, R558H, | CA13-8119 | SEQ ID NO: |
| G561D | | 42, 43 |
| CJL-8109_leuA_N245S, P247C, R558H, | CJL-8120 | SEQ ID NO: |
| G561D | | 44, 45 |
| CJL-8109_leuA_I588P, P247C, R558H, | CJL-8121 | SEQ ID NO: |
| G561D | | 46, 47 |
| CJL-8109_leuA_S211L, P247C, R558H, | CJL-8122 | SEQ ID NO: |
| G561D, I588P | | 48, 49 |
| CJL-8109_leuA_L138G, H162E, S211L, | CJL-8123 | SEQ ID NO: |
| N245S, P247C, R558H, G561D | | 50, 51 |
| CJL-8109_leuA_L138G, H162E, S211L, | CJL-8125 | SEQ ID NO: |
| N245S, P247C, R558H, G561D, I588P | | 52, 53 |

Thereafter, L-leucine producing ability of the wild-type Corynebacterium glutamicum ATCC13032, the prepared CJL-8109, CJL-8117, CJL-8118, CA13-8119, CJL-8120, CJL-8121. CJL-8122, CJL-8123, and CJL-8125 strains was evaluated. In detail, a flask culture was performed by the method of Example 2-1. After completion of the culture, L-leucine productions of the parent strain and the variant strains were measured by HPLC, and the results are shown in Table 11 below.

TABLE 11

| Name of strain | L-leucine (g/L) |
|---|---|
| ATCC13032 | 0.87 |
| CJL-8109 | 2.89 |
| CJL-8117 | 3.55 |
| CJL-8118 | 3.67 |
| CA13-8119 | 4.03 |
| CJL-8120 | 3.46 |
| CJL-8121 | 3.48 |
| CJL-8122 | 4.52 |
| CJL-8123 | 4.02 |
| CJL-8125 | 4.01 |

As shown in Table 11, CJL-8117, CJL-8118, CA13-8119, CJL-8120, CJL-8121, CJL-8122, CJL-8124, and CJL-8125 which are L-leucine producing strains having additional variation of L138G, H162E, S211L, N245S, 1588P, S211L/1588P, L138G/H162E/S211L/N245S, or L138G/H162E/S211L/N245S/1588P in the leuA gene showed about 4- to 5-fold improvement in the L-leucine productivity, as compared to the parent strain, wild-type Corynebacterium glutamicum ATCC13032. It was also confirmed that the L-leucine producing strains, Corynebacterium glutamicum CJL-8117, CJL-8118, CA13-8119, CJL-8120, CJL-8121, CJL-8122, CJL-8123, and CJL-8125 showed about 1.2- to 1.6-fold improvement in the L-leucine productivity, as compared to the parent strain Corynebacterium glutamicum CJL-8109.

These results indicate that the amino acids at positions 138, 162, 211, 245, and 588 of the amino acid sequence of LeuA protein are important sites for the L-leucine productivity.

4-4. Measurement of Isopropylmalate Synthase Activity in LeuA Variant-Introduced Strain

In order to measure the isopropylmalate synthase activity in CJL-8109 and CJL-8117, CJL-8118, CA13-8119, CJL-8120, CJL-8121, CJL-8122, CJL-8123, and CJL-8125 which are the L-leucine-producing strains prepared in Example 4-3, the experiment was performed in the following manner.

Each one platinum loop of the strains (CJL-8109, CJL-8117, CJL-8118, CA13-8119, CJL-8120, CJL-8121, CJL-8122, CJL-8123, CJL-8125) and the wild-type Corynebacterium glutamicum ATCC13032 was inoculated into a 250 ml corner-baffle flask containing 25 ml of a production medium of Table 2, and then cultured at 30° C. for 16 hours under shaking at 200 rpm. After completion of the culture, each culture medium was centrifuged and the supernatant was discarded. The pellet was washed and suspended with a lysis buffer, and disrupted. Protein quantification of the lysate was performed according to the Bradford assay, and the lysate containing 100 μg/ml of protein was used. At this time, absorbance change at 412 nm due to thionitrobenzoate (TNB) formed from DTNB (5,5′-dithiobis-(2-nitrobenzoic acid), Ellman's reagent) by reduction using the produced CoA was measured to determine the activity of isopropylmalate synthase enzyme. The results of measuring the activity of isopropylmalate synthase in each strain are shown in Table 12 below.

TABLE 12

| | Relative activity of |
|---|---|
| Strain | isopropylmalate synthase (%) |
| ATCC13032 | 100 |
| CJL-8109 | 118 |
| CJL-8117 | 121 |
| CJL-8118 | 125 |
| CA13-8119 | 138 |
| CJL-8120 | 122 |
| CJL-8121 | 130 |
| CJL-8122 | 132 |
| CJL-8123 | 135 |
| CJL-8125 | 136 |

Next, in order to examine the degree of release of the feedback inhibition of the enzyme by leucine, the activity of isopropylmalate synthase was determined by measuring CoA generated when the lysate containing 100 μg/ml of protein was used under the condition where 2 g/L of leucine was added. The results of measuring the activity of isopropylmalate synthase in each strain are shown in Table 13 below.

TABLE 13

| | |
|---|---|
| 0 g/l of leucine | 2 g/l of leucine |

| | |
|---|---|
| Strain | Relative activity of isopropylmalate synthase (%) |

| | | |
|---|---|---|
| ATCC13032 | 100 | 36 |
| CJL-8109 | 100 | 83 |
| CJL-8117 | 100 | 83 |
| CJL-8118 | 100 | 83 |
| CA13-8119 | 100 | 93 |
| CJL-8120 | 100 | 85 |
| CJL-8121 | 100 | 88 |
| CJL-8122 | 100 | 92 |
| CJL-8123 | 100 | 90 |
| CJL-8125 | 100 | 91 |

As shown in Tables 12 and 13, CJL-8109 and CJL-8117, CJL-8118, CA13-8119, CJL-8120, CJL-8121, CJL-8122, CJL-8123, and CJL-8125 which are L-leucine producing strains transformed with the LeuA variant-expressing vector showed about 1.18- to 1.38-fold improvement in the activity of isopropylmalate synthase, as compared to the control wild-type Corynebacterium glutamicum ATCC 13032. It was also confirmed that the L-leucine producing strains maintained 83% to 93% of the isopropylmalate synthase activity even under condition where 2 g/L of leucine was added, indicating that feedback inhibition by leucine was released.

CA13-8119 was deposited at the Korean Culture Center of Microorganisms, an international depository authority under the Budapest Treaty, on Feb. 8, 2021, and assigned Accession No. KCCM12949P.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. A polypeptide variant comprising one or more substitutions selected from the group consisting of i) a substitution of an amino acid residue corresponding to position 138 with another amino acid residue, ii) a substitution of an amino acid residue corresponding to position 162 with another amino acid residue, iii) a substitution of an amino acid residue corresponding to position 211 with another amino acid residue, iv) a substitution of an amino acid residue corresponding to position 245 with another amino acid residue, and v) a substitution of an amino acid residue corresponding to position 588 with another amino acid residue, in an amino acid sequence of SEQ ID NO: 1.

2. The polypeptide variant of claim 1, wherein i) leucine, which is the amino acid residue corresponding to position 138, is substituted with glycine.

3. The polypeptide variant of claim 1, wherein ii) histidine, which is the amino acid residue corresponding to position 162, is substituted with glutamate.

4. The polypeptide variant of claim 1, wherein iii) serine, which is the amino acid residue corresponding to position 211, is substituted with leucine.

5. The polypeptide variant of claim 1, wherein iv) asparagine, which is the amino acid residue corresponding to position 245, is substituted with serine.

6. The polypeptide variant of claim 1, wherein v) isoleucine, which is the amino acid residue corresponding to position 588, is substituted with proline.

7. The polypeptide variant of claim 1, comprising any one or more amino acid sequences selected from the group consisting of SEQ ID NO: 6, SEQ ID NO: 8, SEQ ID NO: 10, SEQ ID NO: 12, and SEQ ID NO: 14.

8. A polynucleotide encoding the polypeptide variant of claim 1.

9. A vector comprising the polynucleotide of claim 8.

10. A microorganism of the genus Corynebacterium producing L-leucine, the microorganism comprising the polypeptide variant of claim 1; a polynucleotide encoding the same; or a vector including the same.

11. The microorganism of the genus Corynebacterium of claim 10, wherein the microorganism of the genus Corynebacterium is Corynebacterium glutamicum.

12. A method of producing L-leucine, the method comprising a step of culturing, in a medium, a microorganism of the genus Corynebacterium producing L-leucine, the microorganism comprising the polypeptide variant of claim 1; a polynucleotide encoding the same; or a vector including the same.

13. The method of claim 12, further comprising a step of recovering L-leucine from the medium or from the microorganism after the step of culturing.

14. A composition for producing L-leucine, the composition comprising a microorganism of the genus Corynebacterium producing L-leucine, the microorganism comprising the polypeptide variant of claim 1, a polynucleotide encoding the same, or a vector including the same; or a medium in which the microorganism is cultured.

15. The polypeptide variant according to claim 1, wherein the polypeptide has an isopropylmalate synthase activity.

16. The polypeptide variant according to claim 1, comprising at least 80% sequence identity to SEQ ID NO: 1.


SEQUENCE LISTING (55 sequences)

SEQ ID NO: 1 - protein, 616 aa, Unknown
  leuA(616 aa)
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 2 - DNA, 1851 bp, Unknown
  leuA WT(1851 bp)
atgtctcctaacgatgcattcatctccgcacctgccaagatcgaaaccccagttgggcctcgcaacgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggcttcaaagaaatcgaggtcggtttcccttcagcttcccagactgattttgatttcgttcgtgagatcatcgaaaagggcatgatccctgacgatgtcaccattcaggttctggttcaggctcgtgagcacctgattcgccgtacttttgaagcttgcgaaggcgcaaaaaacgttatcgtgcacttctacaactccacctccatcctgcagcgcaacgtggtgttccgcatggacaaggtgcaggtgaagaagctggctaccgatgccgctgaactaatcaagaccatcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggtgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccgcggcaacggcccactggccgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 3 - DNA, 21 bp, Artificial Sequence
  Primer 1
tatgcttcaccacatgacttc

SEQ ID NO: 4 - DNA, 23 bp, Artificial Sequence
  Primer 2
aaatcatttgagaaaactcgagg

SEQ ID NO: 5 - protein, 581 aa, Unknown
  leuA(581 aa)
MPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 6 - protein, 616 aa, Artificial Sequence
  L138G
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 7 - DNA, 1851 bp, Artificial Sequence
  L138G
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttggggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 8 - protein, 616 aa, Artificial Sequence
  H162E
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVEFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 9 - DNA, 1851 bp, Artificial Sequence
  H162E
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtggagttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 10 - protein, 616 aa, Artificial Sequence
  S211L
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVEFYNSTSILQRNVVFRMDKVQVKKLATDAAELIKTIAQDYPDTNWRWQYLPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 11 - DNA, 1851 bp, Artificial Sequence
  S211L
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtaccttcctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 12 - protein, 616 aa, Artificial Sequence
  I588P
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVEFYNSTSILQRNVVFRMDKVQVKKLATDAAELIKTIAQDYPDTNWRWQYLPESFTGTEVEYAKEVVDAVVEVMDPTPENPMIISLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSPTYASLKAVTSAVNRALDVNHEAVLAGGV

SEQ ID NO: 13 - DNA, 1851 bp, Artificial Sequence
  I588P
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctcccccacctacgcttcgctgaaggcagtgacctccgccgtaaaccgcgcgctggacgtcaaccacgaggcagtcctggctggcggcgtttaa

SEQ ID NO: 14 - protein, 616 aa, Artificial Sequence
  N245S
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVEFYNSTSILQRNVVFRMDKVQVKKLATDAAELIKTIAQDYPDTNWRWQYLPESFTGTEVEYAKEVVDAVVEVMDPTPENPMIISLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 15 - DNA, 1851 bp, Artificial Sequence
  N245S
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatctccctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 16 - DNA, 42 bp, Artificial Sequence
  Primer 3
ggtcgactctagaggatcccctatgcttcaccacatgacttc

SEQ ID NO: 17 - DNA, 42 bp, Artificial Sequence
  Primer 4
caggtgctcacgagcctgaaccccaacctgaatggtgacatc

SEQ ID NO: 18 - DNA, 39 bp, Artificial Sequence
  Primer 5
gacgatgtcaccattcaggttggggttcaggctcgtgag

SEQ ID NO: 19 - DNA, 45 bp, Artificial Sequence
  Primer 6
gtgaattcgagctcggtacccaaatcatttgagaaaactcgaggc

SEQ ID NO: 20 - DNA, 43 bp, Artificial Sequence
  Primer 7
gatggaggttgagttgtagaactccacgataacgttttttgcg

SEQ ID NO: 21 - DNA, 42 bp, Artificial Sequence
  Primer 8
ggcgcaaaaaacgttatcgtggagttctacaactcaacctcc

SEQ ID NO: 22 - DNA, 38 bp, Artificial Sequence
  Primer 9
agtgccggtgaaggactcaggaaggtactgccagcgcc

SEQ ID NO: 23 - DNA, 39 bp, Artificial Sequence
  Primer 10
accaactggcgctggcagtaccttcctgagtccttcacc

SEQ ID NO: 24 - DNA, 42 bp, Artificial Sequence
  Primer 11
catctcaacggtggaacacagggagatgatcattgggttctc

SEQ ID NO: 25 - DNA, 37 bp, Artificial Sequence
  Primer 12
cctgagaacccaatgatcatctccctgtgttccaccg

SEQ ID NO: 26 - DNA, 38 bp, Artificial Sequence
  Primer 13
tgccttcagcgaagcgtaggtgggggagccagcgatgc

SEQ ID NO: 27 - DNA, 39 bp, Artificial Sequence
  Primer 14
ggcgtcggcatcgctggctcccccacctacgcttcgctg

SEQ ID NO: 28 - DNA, 22 bp, Artificial Sequence
  Primer 15
aacacgaccggcatcccgtcgc

SEQ ID NO: 29 - DNA, 23 bp, Artificial Sequence
  Primer 16
aaatcatttgagaaaactcgagg

SEQ ID NO: 30 - DNA, 19 bp, Artificial Sequence
  Primer 23
gtcacccgatcgtctgaag

SEQ ID NO: 31 - DNA, 18 bp, Artificial Sequence
  Primer 24
gtcttaaaaccggttgat

SEQ ID NO: 32 - DNA, 42 bp, Artificial Sequence
  gltA M312I Up F
gtgaattcgagctcggtacccgcgggaatcctgcgttaccgc

SEQ ID NO: 33 - DNA, 42 bp, Artificial Sequence
  gltA M312I Up R
tgtaaacgcggtgtccgaagccgatgaggcggacgccgtctt

SEQ ID NO: 34 - DNA, 42 bp, Artificial Sequence
  gltA M312I Down F
aagacggcgtccgcctcatcggcttcggacaccgcgtttaca

SEQ ID NO: 35 - DNA, 42 bp, Artificial Sequence
  gltA M312I Down R
ggtcgactctagaggatccccttagcgctcctcgcgaggaac

SEQ ID NO: 36 - DNA, 19 bp, Artificial Sequence
  Primer 25
caatgctggctgcgtacgc

SEQ ID NO: 37 - DNA, 20 bp, Artificial Sequence
  Primer 26
ctcctcgcgaggaaccaact

SEQ ID NO: 38 - protein, 616 aa, Artificial Sequence
  L138G, P247C, R558H, G561D
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSITYASLKAVTSAVNRALDVNHEAVLAGGV

SEQ ID NO: 39 - DNA, 1851 bp, Artificial Sequence
  L138G, P247C, R558H, G561D
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttggggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 40 - protein, 616 aa, Artificial Sequence
  H162E, P247C, R558H, G561D
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVEFYNSTSILQRNVVFRMDKVQVKKLATDAAELIKTIAQDYPDTNWRWQYSPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSITYASLKAVTSAVNRALDVNHEAVLAGGV

SEQ ID NO: 41 - DNA, 1851 bp, Artificial Sequence
  H162E, P247C, R558H, G561D
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtggagttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 42 - protein, 616 aa, Artificial Sequence
  S211L, P247C, R558H, G561D
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYLPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSITYASLKAVTSAVNRALDVNHEAVLAGGV

SEQ ID NO: 43 - DNA, 1851 bp, Artificial Sequence
  S211L, P247C, R558H, G561D
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtaccttcctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 44 - protein, 616 aa, Artificial Sequence
  N245S, P247C, R558H, G561D
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIISLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSITYASLKAVTSAVNRALDVNHEAVLAGGV

SEQ ID NO: 45 - DNA, 1851 bp, Artificial Sequence
  N245S, P247C, R558H, G561D
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatctccctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 46 - protein, 616 aa, Artificial Sequence
  I588P, P247C, R558H, G561D
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSPTYASLKAVTSAVNRALDVNHEAVLAGGV

SEQ ID NO: 47 - DNA, 1851 bp, Artificial Sequence
  I588P, P247C, R558H, G561D
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctcccccacctacgcttcgctgaaggcagtgacctccgccgtaaaccgcgcgctggacgtcaaccacgaggcagtcctggctggcggcgtttaa

SEQ ID NO: 48 - protein, 616 aa, Artificial Sequence
  S211L,  P247C, R558H, G561D, I588P
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYLPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSPTYASLKAVTSAVNRALDVNHEAVLAGGV

SEQ ID NO: 49 - DNA, 1851 bp, Artificial Sequence
  S211L,  P247C, R558H, G561D, I588P
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttctggttcaggctcgtgagcacctgattcgccgtacttttgaagcttgcgaaggcgcaaaaaacgttatcgtgcacttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtaccttcctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctcccccacctacgcttcgctgaaggcagtgacctccgccgtaaaccgcgcgctggacgtcaaccacgaggcagtcctggctggcggcgtttaa

SEQ ID NO: 50 - protein, 616 aa, Artificial Sequence
  L138G, H162E, S211L, N245S, P247C, R558H, G561D
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVEFYNSTSILQRNVVFRMDKVQVKKLATDAAELIKTIAQDYPDTNWRWQYLPESFTGTEVEYAKEVVDAVVEVMDPTPENPMIISLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSITYASLKAVTSAVNRALDVNHEAVLAGGV

SEQ ID NO: 51 - DNA, 1851 bp, Artificial Sequence
  L138G, H162E, S211L, N245S, P247C, R558H, G561D
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttggggttcaggctcgtgagcacctgattcgccgtacttttgaagcttgcgaaggcgcaaaaaacgttatcgtggagttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtaccttcctgagtccttcaccggcactgaggttgagtacgccaaggaagttgtggacgcagttgttgaggtcatggatccaactcctgagaacccaatgatcatctccctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 52 - protein, 616 aa, Artificial Sequence
  L138G, H162E, S211L, N245S, P247C, R558H, G561D, I588P
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVGVQAREHLIRRTFEACEGAKNVIVEFYNSTSILQRNVVFRMDKVQVKKLATDAAELIKTIAQDYPDTNWRWQYLPESFTGTEVEYAKEVVDAVVEVMDPTPENPMIISLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSPTYASLKAVTSAVNRALDVNHEAVLAGGV

SEQ ID NO: 53 - DNA, 1851 bp, Artificial Sequence
  L138G, H162E, S211L, N245S, P247C, R558H, G561D, I588P
atgtctcctaacgatgcattcatctccgcacctgccaagatcgaaaccccagttgggcctcgcaatgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggattcaaggaaatcgaggtcggtttcccttcagcttcccagactgattttgatttcgttcgtgagatcatcgaaaaggacatgatccctgacgatgtcaccattcaggttggggttcaggctcgtgagcacctgattcgccgtacttttgaagcttgcgaaggcgcaaaaaacgttatcgtggagttctacaactcaacctccatcctgcagcgcaacgtggtgttccgcatggacaaggtgcaggtgaagaagctggctaccgatgccgctgaactgatcaagaccgtcgctcaggattacccagacaccaactggcgctggcagtaccttcctgagtccttcaccggcactgaggttgagtacgccaaggaagttgtggacgcagttgttgaggtcatggatccaactcctgagaacccaatgatcatctccctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggcgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccacggcaacggcccactggctgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctcccccacctacgcttcgctgaaggcagtgacctccgccgtaaaccgcgcgctggacgtcaaccacgaggcagtcctggctggcggcgtttaa

SEQ ID NO: 54 - DNA, 45 bp, Artificial Sequence
  Primer 27
ggtgatcatctcaacggtggaacacaggttgatgatcattgggtt

SEQ ID NO: 55 - DNA, 45 bp, Artificial Sequence
  Primer 28
aacccaatgatcatcaacctgtgttccaccgttgagatgatcacc